(19) United States
(12) Patent Application Publication
(10) Pub. No.: US 2025/0195602 A1
(43) Pub. Date: Jun. 19, 2025

(54) Compositions and Methods for Treating Neuropathy

(71) Applicant: Creative Bio-Peptides, Inc., Potomac, MD (US)

(72) Inventor: Michael RUFF, Potomac, MD (US)

(21) Appl. No.: 18/559,874

(22) PCT Filed: May 20, 2022
(86) PCT No.: PCT/US2022/030305

§ 371 Date: Nov. 9, 2023

Related U.S. Application Data
(60) Provisional application No. 63/191,010, filed on May 20, 2021.

(51) Int. Cl. A61K 38/08; A61K 9/00; A61K 45/06; A61P 25/02

(52) U.S. Cl. CPC A61K 38/08; A61K 9/0019; A61K 9/0053; A61K 45/06; A61P 25/02

(57) ABSTRACT

Disclosed herein are polypeptides, derivatives thereof, and salts thereof, as well as pharmaceutical compositions containing these, useful alone or in combination with other therapies for treating chemotherapy induced peripheral neuropathy.

### CROSS REFERENCE TO RELATED APPLICATIONS

This application is a National Stage Entry of International Application No. PCT/US2022/030305, filed May 20, 2022, which claims the benefit of U.S. Provisional Application No. 63/191,010, filed May 20, 2021, the disclosures of which are incorporated herein by reference in their entirety.

### STATEMENT AS TO FEDERALLY SPONSORED RESEARCH

This invention was made with government support under Contract number MCDC-18-03-001 awarded by Department of Defense and Contract number 1R44NS120541-01 awarded by Department of Health and Human Services. The government has certain rights in the invention.

### INCORPORATION BY REFERENCE

All publications, patents, and patent applications herein are incorporated by reference to the same extent as if each individual publication, patent, or patent application was specifically and individually indicated to be incorporated by reference. In the event of a conflict between a term herein and a term in an incorporated reference, the term herein controls.

### SUMMARY

Disclosed herein are methods of treating a chemotherapy induced peripheral neuropathy (CIPN) in a subject in need thereof. In some embodiments, the method comprises administering to the subject a therapeutically effective amount of a pharmaceutical composition to treat CIPN. In some embodiments, the pharmaceutical composition can comprise: (a) a polypeptide, a derivative thereof, or a salt thereof, wherein the polypeptide comprises at least five contiguous amino acids or derivatives thereof comprising the general formula: E-F-G-H-I, wherein: E can be D-Ser, D-Thr, D-Asn, D-Glu, D-Arg, D-Ile, or D-Leu, or a derivative of any of these; F can be D-Ser, D-Thr, D-Asp, or D-Asn, or a derivative of any of these; G can be D-Thr, D-Ser, D-Asn, D-Arg, D-Gln, D-Lys, or D-Trp, or a derivative of any of these; H can be D-Tyr, or a derivative thereof; and I can be D-Thr, D-Ser, D-Arg, or Gly, or a derivative of any of these. In some embodiments, the polypeptide, the derivative thereof, or the salt thereof can comprise at least eight contiguous amino acids or derivatives thereof, comprising the general formula A-B-C-E-F-G-H-I, and wherein: A can be D-Ala, or a derivative thereof; B can be D-Ser, or D-Thr, or a derivative of any of these; C can be D-Ser, or D-Thr, or a derivative of any of these; E can be D-Ser, D-Thr, D-Asn, D-Glu, D-Arg, D-Ile, or D-Leu, or a derivative of any of these; F can be D-Ser, D-Thr, D-Asp, or D-Asn, or a derivative of any of these; G can be D-Thr, D-Ser, D-Asn, D-Arg, D-Gln, D-Lys, or D-Trp, or a derivative of any of these; H can be D-Tyr, or a derivative thereof; and I can be D-Thr, D-Ser, D-Arg, or Gly or a derivative of any of these. In some embodiments, the polypeptide or the salt thereof can be D-Thr, D-Thr, D-Asn, D-Tyr, and D-Thr or a salt thereof. In some embodiments, the derivative of I can be esterified, glycosylated, or amidated at the C terminus. In some embodiments, the pharmaceutical composition can be in unit dose form. In some embodiments, the pharmaceutical composition can further comprise an excipient, a diluent, a carrier, or a combination thereof. In some embodiments, CIPN can be accompanied or preceded by a nerve injury, an inflammation, a pain or a combination thereof. In some embodiments, the administering can be daily, weekly, or monthly. In some embodiments, administering can be once, twice, three, or four times per day. In some embodiments, the pharmaceutical composition can be administered for about: one day, two days, three days, four days, five days, six days, one week, two weeks, three weeks, four weeks, five weeks, one month, two months, three months, four months, five months, six months, seven months, eight months, nine months, ten months, eleven months, one year, two years, or for life. In some embodiments, the pharmaceutical composition comprises the polypeptide, the derivative thereof, or the salt thereof in an amount of from about 0.005 mg to about 1000 mg. In some embodiments, the pharmaceutical composition can be administered by: an oral route, an injection route, a sublingual route, a buccal route, a rectal route, a vaginal route, an ocular route, an otic route, a nasal route, an internasal route, an inhalation route, a cutaneous route, a subcutaneous route, an intramuscular route, an intravenous route, a systemic route, a local route, a systemic route, a transdermal route, or any combination thereof. In some embodiments, the pharmaceutical composition can be formulated for oral administration. In some embodiments, the pharmaceutical composition can be in a form of a pill or a liquid. In some embodiments, a second therapy can be administered concurrently or consecutively. In some embodiments, the second therapy can comprise a gabapentinoid, an opioid, a voltage-gated sodium channel inhibitor, an anti-nerve growth factor, an nonsteroidal anti-inflammatory drug, aspirin, a corticosteroid, acetaminophen, a muscle relaxant, an anti-anxiety drug, an antidepressant, a cox-2 inhibitor, a local anesthetic, an anticonvulsant, a cannabinoid, an NMDA receptor antagonist, an α2-adrenergic receptor agonist or any combination thereof. In some embodiments, the pharmaceutical composition can further comprise the second therapy. In some embodiments, the subject can be diagnosed with the CIPN prior to the administration. In some embodiments, the subject can be diagnosed with pain prior to the administration. In some embodiments, the diagnoses can comprise an in vitro test, a physical exam, an imaging diagnostic or a combination thereof. In some embodiments, the CIPN can be associated with administration of a platinum-based chemotherapy drug. In some embodiments, the platinum-based chemotherapy drug can be cisplatin, carboplatin, oxaliplatin, a salt of any of these, or a derivative of any of these. In some embodiments, the CIPN can be associated with administration of a taxane. In some embodiments, the taxane can be docetaxel, paclitaxel, cabazitaxel, a salt of any of these, or a derivative of any of these. In some embodiments, the CIPN can be associated with administration of an epothilone. In some embodiments, the epothilone can be ixabepilone or a salt thereof. In some embodiments, the CIPN can be associated with administration of a plant alkaloid. In some embodiments, the plant alkaloid can be vinblastine, vincristine, vinorelbine, etoposide, a salt of any of these, or a derivative of any of these. In some embodiments, the CIPN can be associated with administration of a thalidomide, a lenalidomide, a pomalidomide, a salt of any of these, or a derivative of any of these. In some embodiments, the CIPN can be associated with administration of a proteasome inhibitor. In some embodiments, the proteasome inhibitor can be a bortezomib, a carfilzomib, a salt of any of these, or a derivative of any of these. In some embodiments, the subject can be a mammal. In some embodiments, the mammal can be a human. In some embodiments, the pharmaceutical composition can treat CIPN at a dose 40-fold lower weight-to-weight than morphine in a rat animal model.

### BRIEF DESCRIPTION OF THE DRAWINGS

FIG. 1 shows the ordinal pain scale, which is based on the subjective ranking of pain by a patient or subject.

FIG. 2 shows graphs of the pharmacokinetic properties of RAP-103 (R103). FIG. 2A shows the pharmacokinetic properties of RAP-103 with rapid brain entry by oral and intravenous dosing in rats and guinea pigs. FIG. 2B shows the plasma levels of RAP-103 for hours in non-human primates (monkeys).

FIG. 3 shows an exemplary peptide manufacturing process for RAP-103.

FIG. 4 shows a graph of the increased paw withdraw threshold for rats treated with paclitaxel to induce chronic pain for the effect of RAP-103 compared to vehicle. Morphine was used as a positive control for pain response.

### DETAILED DESCRIPTION

### Overview

Disclosed herein are non-opioid compositions, pharmaceutical formulations, methods of treatment of a condition or disease, a diagnostic to detect the condition or disease, or a kit comprising a pharmaceutical formulation. These compositions or formulations more specifically concern subjects exhibiting pain due at least in part from peripheral and central neuropathic pain.

In certain instances, the treatment is for chemotherapy induced peripheral neuropathy. Chemotherapy-induced peripheral neuropathy (CIPN) is a dose-limiting side effect of many anticancer drugs, such as cisplatin, oxaliplatin, paclitaxel, thalidomide, bortezomib, and vincristine. CIPN is considered a severe side effect of therapeutic agents with limited treatment options. Approximately one third of all patients treated with chemotherapy for cancer develop CIPN. The prevalence of CIPN was found to be 68% within the first month of chemotherapy treatment to as high as 96.2% depending on the chemotherapeutic agent and the type of cancer in which the drug is used.

Different neurochemical changes may occur in primary afferent neurons and in the spinal cord, a site of central pain activation, in different persistent pain states. CIPN creates a unique pain state that is thought to involve sensitization of the nervous system and patients with this type of pain may be difficult to treat. Tumors themselves can cause pain or contribute to CIPN by secreting a variety of factors that sensitize or directly excite primary afferent neurons. Tumors may also compress and directly injure nerves, causing pain. CIPN and cancer pain are distinct pain conditions, resulting from unique neurochemical pain states which are not solely inflammatory or neuropathic, and will require new treatment modalities such as the peptides disclosed herein. The chemotherapy can damage peripheral neurons making them both generators of pain and while at the same time, non-responsive to or less responsive to traditional pain therapies, such as opiates and gabapentin. Because CIPN is unique among peripheral neuropathies, and arises from multiple causes, including damage to peripheral nerves from the chemotherapy, alteration in nerve function as a result of exposure to chemotherapy and factors secreted by a tumor or cancer, and in some cases, potential structural alterations caused by the presence of the tumor or cancer; and because CIPN is refractory to conventional pain treatments, it was thought that the peptides herein would be unlikely to treat CIPN. Surprisingly, in an animal model of CIPN, a representative peptide herein (RAP-103) was at least equivalent in treating paclitaxel induced CIPN—at a dose that was about 40-fold lower on a weight basis—than a standard of care (morphine). This superior result was unexpected for all the reasons described above.

In some instances, the peptides herein can (treat) reduce CIPN arising from multiple causes, e.g., tumor and chemotherapy, and structural insult (tumor compressing a nerve) and even more remarkably, do so at doses that are at least about 40-fold lower than a conventional therapy on a weight-to-weight basis.

In some instances, the peptides herein can reduce pain at a dose 5-fold to about 40-fold lower (weight-to-weight) than a commonly administered pain medication (e.g., an opioid or another conventional therapy). In some instances, the peptides herein can reduce pain at a dose 5-fold to about 35-fold lower (weight-to-weight) than a commonly administered pain medication. In some instances, the peptides herein can reduce pain at a dose 5-fold to about 30-fold lower (weight-to-weight) than a commonly administered pain medication. In some instances, the peptides herein can reduce pain at a dose 5-fold to about 25-fold lower (weight-to-weight) than a commonly administered pain medication. In some instances, the peptides herein can reduce pain at a dose 5-fold to about 20-fold lower (weight-to-weight) than a commonly administered pain medication. In some instances, the peptides herein can reduce pain at a dose 5-fold to about 15-fold lower (weight-to-weight) than a commonly administered pain medication. In some instances, the peptides herein can reduce pain at a dose 5-fold to about 10-fold lower (weight-to-weight) than a commonly administered pain medication. In some instances, the peptides herein can reduce pain at a dose of about: 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold lower (weight-to-weight) than a commonly administered pain medication.

In certain instances, the conventional therapy is an opiate. In certain instances, the opiate is morphine.

Chemotherapy-induced peripheral neuropathy (CIPN) is a common and potentially dose-limiting side effect of many chemotherapy drugs. The incidence of CIPN in the cancer population depends upon the agent used to treat but has been estimated to be as high as 90% in patients treated with any neurotoxic chemotherapeutic agents. The pain and loss of function that result from CIPN are debilitating and can impact all domains of quality of life.

While not limiting, several chemotherapeutic drugs and classes thereof have been known to be associated with CIPN. Examples of such drugs include: platinum drugs like cisplatin, carboplatin, and oxaliplatin; taxanes including paclitaxel, docetaxel, and cabazitaxel; epothilones, such as ixabepilone; plant alkaloids, such as vinblastine, vincristine, vinorelbine; and etoposide, thalidomide, lenalidomide, and pomalidomide, bortezomib, carfilzomib, and eribulin. In some cases, the CIPN is not associated with administration of a streptozotocin or a salt thereof.

In certain instances, the peptides herein can reduce pain by about: 2-fold, 3-fold, 4-fold, 5-fold, 6-fold, 7-fold, 8-fold, 9-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 26-fold, 27-fold, 28-fold, 29-fold, 30-fold, 31-fold, 32-fold, 33-fold, 34-fold, 35-fold, 36-fold, 37-fold, 38-fold, 39-fold, 40-fold, 41-fold, 42-fold, 43-fold, 44-fold, 45-fold, 46-fold, 47-fold, 48-fold, 49-fold, or 50-fold on a weight-to-weight basis as compared to a conventional pain management therapy when the chemotherapy resulting in CIPN is cisplatin, carboplatin, oxaliplatin, docetaxel, paclitaxel, cabazitaxel, ixabepilone, vinblastine, vincristine, vinorelbine, etoposide, thalidomide, lenalidomide, pomalidomide, bortezomib, carfilzomib, or a derivative of any of these. In some instances, a peptide herein may comprise up to eight or more than eight contiguous amino acids or derivatives thereof, comprising the general formula A-B-C-E-F-G-H-I, and wherein: A is D-Ala, or a derivative thereof; B is D-Ser, or D-Thr, or a derivative of any of these; C is D-Ser, or D-Thr, or a derivative of any of these; E is D-Ser, D-Thr, D-Asn, D-Glu, D-Arg, D-Ile, or D-Leu, or a derivative of any of these; F is D-Ser, D-Thr, D-Asp, or D-Asn, or a derivative of any of these; G is D-Thr, D-Ser, D-Asn, D-Arg, D-Gln, D-Lys, or D-Trp, or a derivative of any of these; H is D-Tyr, or a derivative thereof, and I is D-Thr, D-Ser, D-Arg, or Gly or a derivative of any of these. In some cases, a peptide herein can comprise at least five contiguous amino acids or derivatives thereof comprising the general formula: E-F-G-H-I, wherein: E is D-Ser, D-Thr, D-Asn, D-Glu, D-Arg, D-Ile, or D-Leu, or a derivative of any of these; wherein F is D-Ser, D-Thr, D-Asp, or D-Asn, or a derivative of any of these; wherein G is D-Thr, D-Ser, D-Asn, D-Arg, D-Gln, D-Lys, or D-Trp, or a derivative of any of these; wherein H is D-Tyr, or a derivative thereof; and wherein I is D-Thr, D-Ser, D-Arg, or Gly, or a derivative of any of these. In some cases, a conventional pain management therapy can comprise a gabapentinoid, an opioid, a voltage-gated sodium channel inhibitor, an anti-nerve growth factor, an nonsteroidal anti-inflammatory drug, aspirin, a corticosteroid, acetaminophen, a muscle relaxant, an anti-anxiety drug, an antidepressant, a cox-2 inhibitor, a local anesthetic, an anticonvulsant, a cannabinoid, an NMDA receptor antagonist, an α2-adrenergic receptor agonist or any combination thereof.

In other instances, the compositions, pharmaceutical formulations, and treatments are for other forms of peripheral neuropathy.

Peptides in the compositions and pharmaceutical formulations disclosed herein may be used for treatment in a subject to block the innate immune chemokine receptors that when activated promote pain, block the action of opioids to stop pain, and damage nerves, causing even more pain. The peptides provided herein provide a safe and effective, non-opioid treatment for neuropathic pain conditions such as chemotherapy induced peripheral neuropathy.

Multiple diverse mechanisms can be active in CIPN which distinguish this complex pain condition from other neuropathic pain states. The dorsal root ganglia lacks an efficient blood-brain barrier and is prone to neurotoxic damage by chemo-agents including DNA damage and modifications of neurofilaments. Vincristine and paclitaxel can increase pro-inflammatory cytokines in peripheral nerves, which is damaging. Tumors themselves secrete a variety of cytokines and factors that sensitize or directly excite primary afferent neurons, causing the sensation of pain. The cytokines IL-1, IL-6, and TNFα can be aberrantly produced by cancer and immune system cells and are of particular relevance in pain. There are distinctions among the types of inflammation (classical inflammation via immune cells, neurogenic inflammation initiated by activation of peripheral nervous system C-fiber neurons rather than by an immunological event, and neuroinflammation via activated astrocytes and microglia) that contribute to pain. CIPN can be a chronic pain condition which can result from all three of these types of inflammation or a mixture of the types of inflammation described above, which the peptides disclosed herein can treat, in addition to blocking the contributions of chemotherapy and tumor cell secretions to pain.

The multi-chemokine receptor antagonist peptide RAP-103 (All D-peptide-Thr-Thr-Asn-Tyr-Thr) is being developed to provide a non-opioid, potentially disease modifying treatment for neuropathic pain conditions. Previous work has shown proof-of-concept (POC) animal studies in neuropathic pain by partial nerve ligation and diabetes. In some cases, RAP-103 can be used to treat chemotherapy-induced peripheral neuropathy (CIPN), a dose-limiting side effect of many anticancer drugs that can affect 68% or more of cancer patients. A neuroprotective mechanism for RAP-103 pain effects may be established. Additional chronic pain patients that may benefit from RAP-103 (All-D-TTNYT) include those with spinal cord injury, chronic low-back pain, and post-herpetic neuralgia. A target patient population could be the 2.5 million Americans with diabetic peripheral neuropathy, the most common chronic neuropathic pain condition. Another, and different target patient population, could be those that suffer from CIPN. Chemokines, molecules of the innate immune system that mediate inflammation, acting through receptors such as CCR2, CCR5, and CCR8 can promote pain by multiple mechanisms that cause sustained excitability of primary nociceptive neurons, desensitize endogenous opioid anti-pain effects, activate microglia and astrocytes, and cause peripheral monocyte infiltration into CNS. Blocking multiple chemokine receptors that can establish and sustain chronic pain with the multi-chemokine receptor antagonist (CRA) RAP-103 may be a non-opioid approach to pain treatment. Independent research by others shows the value of CRA's in diverse chronic pain conditions. RAP-103 is a CRA because of its ease of dosing, rapid entry into the CNS, lack of toxicity and side effects, and potential to treat the defining pathology of neuropathic pain, axonal degeneration. Unlike current FDA approved chemokine antagonists (plerixafor, Maraviroc) which have significant safety concerns (allergic risks, need to be injected, “black-box” warning for hepatotoxicity), RAP-103 can be safe and may target multiple chemokine receptors (CCR2/CCR5/CCR8) implicated in pain states. A scale-up peptide manufacture may be conducted and may complete IND-enabling pre-clinical safety and PK/PD studies. In some cases, the peptides disclosed herein can be used to treat neuropathies, like CIPN, and neuralgias, and back pain.

### Definitions

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various aspects may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used herein, the term ‘about’ a number can refer to that number plus or minus 10% of that number. The term ‘about’ a range can refer to that range minus 10% of its lowest value and plus 10% of its greatest value.

As used in the specification and claims, the singular forms “a”, “an” and “the” include plural references unless the context clearly dictates otherwise. For example, the term “a sample” includes a plurality of samples, including mixtures thereof.

The terms “determining”, “measuring”, “evaluating”, “assessing,” “assaying,” and “analyzing” are often used interchangeably herein to refer to forms of measurement and include determining if an element may be present or not (for example, detection). These terms may include quantitative, qualitative or quantitative, and qualitative determinations. Assessing may be alternatively relative or absolute. “Detecting the presence of” includes determining the amount of something present, as well as determining whether it may be present or absent.

The terms “subject,” “individual,” or “patient” are often used interchangeably herein. A “subject” may be a biological entity containing expressed genetic materials. The biological entity may be a plant, animal, or microorganism, including, for example, bacteria, viruses, fungi, and protozoa. The subject may be tissues, cells and their progeny of a biological entity obtained in vivo or cultured in vitro. The subject may be a mammal. The mammal may be a human. The subject may be diagnosed or suspected of being at high risk for a disease. In some cases, the subject may not be necessarily diagnosed or suspected of being at high risk for the disease.

The term “at least partially” may refer to a qualitative condition that exhibits a partial range or degree of a feature or characteristic of interest. For example, at least partially may comprise a reduction in peripheral neuropathy that is at least about: 5%, 10%, 20%, 30%, 40%, 50%, 60%, 70%, 75%, 80%, 85%, 90%, 95%, 99%, or 100% reduced relative to untreated.

The term “in vivo” may be used to describe an event that takes place in a subject's body.

The term “ex vivo” may be used to describe an event that takes place outside of a subject's body. An “ex vivo” assay may not be performed on a subject. Rather, it may be performed upon a sample separate from a subject. An example of an “ex vivo” assay performed on a sample may be an “in vitro” assay.

The term “in vitro” may be used to describe an event that takes place contained in a container for holding laboratory reagent such that it may be separated from the living biological source organism from which the material may be obtained. In vitro assays may encompass cell-based assays in which cells alive or dead are employed. In vitro assays may also encompass a cell-free assay in which no intact cells are employed.

As used herein, the terms “treatment” or “treating” are used in reference to a pharmaceutical or other intervention regimen for obtaining beneficial or desired results in the recipient such as preventing symptoms of peripheral neuropathy from occurring or reducing or eliminating a pain, an inflammation, nerve damage or a combination thereof. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit may be achieved with the eradication or amelioration of one or more of the physiological symptoms associated with the underlying disorder such that an improvement may be observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

As used herein, a “dose” can refer to a measured quantity of a therapeutic agent to be taken at one time.

As used herein, the term “unit dose” or “dosage form” may be used interchangeably and may be meant to refer to pharmaceutical drug products in the form in which they are marketed for use, with a specific mixture of active ingredients and inactive components or excipients, in a particular configuration, and apportioned into a particular dose to be delivered. The term “unit dose” may also sometimes encompass non-reusable packaging, although the FDA distinguishes between unit dose “packaging” or “dispensing”. More than one unit dose may refer to distinct pharmaceutical drug products packaged together, or to a single pharmaceutical drug product containing multiple drugs and/or doses. The term “unit dose” may also sometimes refer to the particles comprising a pharmaceutical composition, and to any mixtures involved. Types of unit doses may vary with the route of administration for drug delivery, and the substance(s) being delivered. A solid unit dose may be the solid form of a dose of a chemical compound used as a pharmaceutically acceptable drug or medication intended for administration or consumption.

As used herein, “pharmaceutically acceptable salt” may refer to pharmaceutical drug molecules, which may be formed as a weak acid or base, chemically made into their salt forms, most frequently as the hydrochloride, sodium, or sulfate salts. Drug products synthesized as salts may enhance drug dissolution, boost absorption into the bloodstream, facilitate therapeutic effects, and increase its effectiveness. Pharmaceutically acceptable salts may also facilitate the development of controlled-release dosage forms, improve drug stability, extend shelf life, enhance targeted drug delivery, and improve drug effectiveness.

The phrase “pharmaceutically acceptable excipient” as used herein may refer to a pharmaceutically acceptable material, composition or vehicle, such as a liquid or solid filler, diluent, excipient, carrier, solvent or encapsulating material.

As used herein, a “pharmaceutical agent” may refer to an agent or a therapy that may be used to prevent, diagnose, treat, or cure a disease, or combinations thereof. In some cases, a pharmaceutical agent can comprise engineered polynucleotide, a DNA encoding the engineered polynucleotide, or a vector containing or encoding the engineered polynucleotide, or, in some aspects, a method described herein may comprise administering a therapeutically effective amount of these to a subject, who can be a human or animal subject, who can be a mammal.

As used herein, “agent” or “biologically active agent” may refer to a biological, pharmaceutical, or chemical compound or a salt of any of these. Non-limiting examples may include a simple or complex organic or inorganic molecule, a peptide, a protein, a nucleotide such as an engineered single stranded RNA, an engineered single stranded DNA, an alternative nucleic acid, a protein, a carbohydrate, a toxin, or a chemotherapeutic compound. Various compounds may be synthesized, for example, small molecules and oligomers (e.g., oligopeptides and oligonucleotides), or synthetic organic compounds based on various core structures. In addition, various natural sources may provide compounds for screening, such as plant or animal extracts, and the like.

As used herein, the terms “effective amount” or “therapeutically effective amount” of a drug used to treat a disease may be an amount that may reduce the severity of a disease, reduce the severity of one or more symptoms associated with the disease or its treatment, or delay the onset of more serious symptoms or a more serious disease that may occur with some frequency following the treated condition. An “effective amount” may be determined empirically and in a routine manner, in relation to the stated purpose.

As used herein, “time to peak plasma concentration” can refer to the time required for a drug to reach peak concentration in plasma. Peak concentration in plasma can be defined as the plasma concentration that a drug achieves in a specified compartment or test area of the body after the drug has been administered and before the administration of a second dose.

The term “substantially” or “essentially” can refer to a qualitative condition that exhibits an entire or nearly total range or degree of a feature or characteristic of interest. In some cases, substantially can refer to a pain level that varies from a mean or median pain level by about plus or minus: 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%. For example, substantially can refer to: 70%, 75%, 80%, 85%, 90%, 95%, 99%, or 100% reduced pain. In some cases, substantially can refer to at least about: 70%, 75%, 80%, 85%, 90%, 95%, 99%, or 100% of the total range or degree of a feature or characteristic of interest.

As used herein, “HPLC” can refer to high-performance liquid chromatography (formerly referred to as high-pressure liquid chromatography), which is a technique in analytical chemistry used to separate, identify, and quantify each component in a mixture. HPLC can be a common technique used in pharmaceutical development, as it can be a method to ensure product purity.

The terms peptide and polypeptide can be used interchangeably herein.

The term “fragment,” as used herein, may be a portion of a sequence, a subset that may be shorter than a full-length sequence. A fragment may be a portion of a gene. A fragment may be a portion of a peptide or protein. A fragment may be a portion of an amino acid sequence. A fragment may be a portion of an oligonucleotide sequence. A fragment may be less than about: 20, 30, 40, 50 amino acids in length. A fragment may be about 10%, about 15%, about 20%, about 25%, about 30%, about 35%, about 40%, about 50%, about 60% or about 70% of the total length of an amino acid sequence or a nucleotide sequence. A fragment may be less than about: 20, 30, 40, 50 oligonucleotides in length.

As used herein, amino acids can be referenced by their one or three letter codes, which are shown in Table 1.

TABLE 1
Amino acids

| | | |
|---|---|---|
| Alanine | Ala | A |
| Arginine | Arg | R |
| Asparagine | Asn | N |
| Aspartic Acid | Asp | D |
| Cysteine | Cys | C |
| Glutamic Acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Amino acids, depending upon the configuration at the alpha carbon, can be D or L-excepting glycine which does not contain four non-identical substituents on its alpha carbon atom. The designations D and L should not be confused with one letter amino acid codes. In some embodiments, D amino acids are designated with a D in front of the amino acid (e.g., D-Ser, dA). If the amino acid has four non-identical substituents on its alpha carbon atom, and the amino acid is not designated with a D in front of the amino acid, the amino acid can be of the L configuration. The amino acid glycine, lacking four non-identical substituents on its alpha carbon atom, may not be D or L.

The section headings used herein are for organizational purposes only and are not to be construed as limiting the subject matter described.

Peptides herein can include peptides in Table 2.

TABLE 2
Peptide sequences
Peptide Sequences

TTNYT (RAP-103)
SSTYR
STNYT
NTSYG
dASTTTNYT-NH2 (DAPTA)
ASTTTNYT (RAP-310)

Wherein in the table, peptides TTNYT, SSTYR, STNYT and ASTTTNYT are all D-peptides such that each amino acid in the peptide is in the D configuration. Wherein in the table, each amino acid of peptide NTSYG, except for glycine, is in the D configuration. And wherein in the table, dASTTTNYT-NH2 the alanine is in the D configuration, and all other amino acids in peptide 5 are in the L configuration.

### Peptide Compositions

In some instances, a composition is provided which comprises a polypeptide wherein the polypeptide comprises at least five contiguous amino acids or derivatives thereof comprising the general formula: E-F-G-H-I, wherein: E is D-Ser, D-Thr, D-Asn, D-Glu, D-Arg, D-Ile, or D-Leu, or a derivative of any of these; wherein F is D-Ser, D-Thr, D-Asp, or D-Asn, or a derivative of any of these; wherein G is D-Thr, D-Ser, D-Asn, D-Arg, D-Gln, D-Lys, or D-Trp, or a derivative of any of these; wherein H is D-Tyr, or a derivative thereof; and wherein I is D-Thr, D-Ser, D-Arg, or Gly, or a derivative of any of these.

In some instances, E can be D-Thr. In some instances, F can be D-Thr. In some instances, E can be D-Thr and F can be D-Thr. In some instances, G can be D-Asn. In some instances, F can be D-Thr and G can be D-Asn. In some instances, E can be D-Thr, F can be D-Thr, and G can be D-Asn. In some instances, H can be D-Tyr. In some instances, E can be D-Thr and H can be D-Tyr. In some instances, G can be D-Asn and H can be D-Tyr. In some instances, I can be D-Thr. In some instances, E can be D-Thr, F can be D-Thr, G can be D-Asn, H can be D-Tyr, and I can be D-Thr.

In some instances, the polypeptide may comprise at least eight contiguous amino acids or derivatives thereof, comprising the general formula A-B-C-E-F-G-H-I, and wherein: A is D-Ala, or a derivative thereof; B is D-Ser, or D-Thr, or a derivative of any of these; C is D-Ser, or D-Thr, or a derivative of any of these; E is D-Ser, D-Thr, D-Asn, D-Glu, D-Arg, D-Ile, or D-Leu, or a derivative of any of these; F is D-Ser, D-Thr, D-Asp, or D-Asn, or a derivative of any of these; G is D-Thr, D-Ser, D-Asn, D-Arg, D-Gln, D-Lys, or D-Trp, or a derivative of any of these; H is D-Tyr, or a derivative thereof, and I is D-Thr, D-Ser, D-Arg, or or a derivative of any of these.

In some cases, A can be D-Ala. In some instances, B can be D-Ser. In some instances, B can be D-Thr. In some instances, A can be D-Ala and B can be D-Ser. In some instances, A can be D-Ala and B can be D-Thr. In some instances, C can be D-Ser. In some instances, C can be D-Thr. In some instances, B can be D-Ser and C can be D-Ser. In some instances, B can be D-Thr and C can be D-Ser. In some instances, B can be D-Thr and C can be D-Thr. In some instances, E can be D-Thr. In some instances, F can be D-Thr. In some instances, E can be D-Thr and F can be D-Thr. In some instances, G can be D-Asn. In some instances, F can be D-Thr and G can be D-Asn. In some instances, E can be D-Thr, F can be D-Thr, and G can be D-Asn. In some instances, H can be D-Tyr. In some instances, E can be D-Thr and H can be D-Tyr. In some instances, G can be D-Asn and H can be D-Tyr. In some instances, I can be D-Thr. In some instances, E can be D-Thr, F can be D-Thr, G can be D-Asn, H can be D-Tyr, and I can be D-Thr.

In some instances, the polypeptide sequence can comprise a sequence of ASTTTNYT, where each amino acid, individually, can be of the L or of the D configuration; in some instances, all amino acids in the sequence ASTTTNYT can be in the L configuration; in some instances, all amino acids int the sequence ASTTTNYT can be of the D configuration.

In some instances, the polypeptide sequence can comprise a sequence of TTNYT, where each amino acid, individually, can be of the L or of the D configuration; in some instances, all amino acids in the sequence TTNYT can be in the L configuration; in some instances, all amino acids in the sequence TTNTY can be of the D configuration. ASTTTNYT-NH2, where each amino acid, individually, can be of the L or of the D configuration; in some instances, all amino acids in the sequence ASTTTNYT-NH₂ can be in the L configuration; in some instances, all amino acids int the sequence ASTTTNYT-NH₂ can be of the D configuration. In some instances, the A in the sequence ASTTTNYT-NH₂ can be in the D configuration and all other amino acids in this sequence can be in the L configuration. In the sequence, the —NH₂ designations that the amino acid threonine in the C terminal end of the sequence has an amide as opposed to a carboxylic acid.

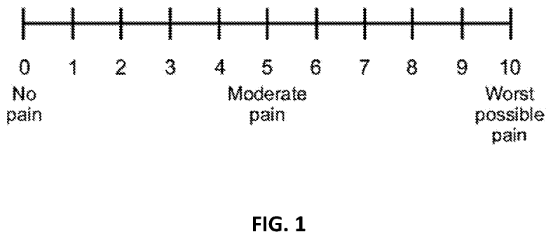

In some instances, the polypeptide sequence is a multi-chemokine receptor antagonist RAP-103 (R103) (All D-peptide-Thr-Thr-Asn-Tyr-Thr) as shown in FIGS. 1-4.

In some instances, the polypeptide described herein can be in the form of a pharmaceutically acceptable salt, such as acetate.

### Pharmaceutical Compositions

An active pharmaceutical ingredient may be any substance or mixture of substances intended to be used in the manufacture of a drug (medicinal) product and that, when used in the production of a drug, becomes an active ingredient of the drug product. Such substances may be intended to furnish pharmacological activity or other direct effect in the diagnosis, cure, mitigation, treatment, or prevention of disease or to affect the structure or function of the body.

### Representative Acids for Addition Salts

In some embodiments, the pharmaceutically acceptable salt of the polypeptide can be formed from the polypeptide and an acid. In some embodiments, the acid can be at least one of: 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic acid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, glycolic acid, hippuric acid, hydrobromic acid, hydrochloric acid, isobutyric acid, lactic acid (DL), lactobionic acid, lauric acid, maleic acid, malic acid (−L), malonic acid, mandelic acid (DL), methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), undecylenic acid, or any combination thereof.

### Representative Salts

In some embodiments, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like. In some instances, a salt of a polypeptide or derivative thereof or a compound can be a Zwitterionic salt.

In some aspects, the pharmaceutical composition comprising the salt of the pharmaceutically active ingredient, wherein the salt comprises an organic salt, an inorganic salt, or any combination thereof. In some cases, an organic salt may comprise a phosphinate (e.g., sodium hypophosphite), a hydrazinium salt, a urate, a diazonium salt, an oxalate salt, a tartrate, a choline chloride. An example of an inorganic salt may be sodium chloride, calcium chloride, magnesium chloride, sodium bicarbonate, potassium chloride, sodium sulfate, calcium carbonate, calcium phosphate, or any combination thereof.

In some aspects, the pharmaceutical composition comprising the salt of the pharmaceutically active ingredient, wherein the salt comprises an HCl salt, an ascorbic acid salt, a mandelic acid salt, an aspartic acid salt, a carbonic acid salt, a citric acid salt, a formic acid salt, a glutamic acid salt, a lactic acid salt, a lauric acid salt, a maleic acid salt, a borate salt, a bitartrate salt, a palmitic acid salt, a phosphoric acid salt, or any combination thereof.

In some aspects, the pharmaceutically acceptable salts include, but are not limited to, metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like; inorganic acid salts such as hydrochloride, hydrobromide, phosphate, sulphate and the like; organic acid salts such as citrate, lactate, tartrate, maleate, fumarate, mandelate, acetate, dichloroacetate, trifluoroacetate, oxalate, formate and the like; sulfonates such as methanesulfonate, benzenesulfonate, p-toluenesulfonate and the like; and amino acid salts such as arginate, asparginate, glutamate and the like.

### Representative Excipients

In some embodiments, the pharmaceutically acceptable carrier, diluent, or excipient, can include pharmaceutically acceptable excipients. As used herein, “excipient” can refer to a substance formulated alongside the active ingredient of a medication, included for the purpose of long-term stabilization, bulking up solid formulations that contain potent active ingredients in small amounts, and/or to confer a therapeutic enhancement on the active ingredient(s) in the final dosage form. Excipients may facilitate drug absorption, reduce viscosity, or enhance solubility. Excipients may also facilitate the handling of the active ingredients, improve in vitro stability, and/or extend pharmaceutical product shelf life. Excipient selection may vary with the route of administration for drug delivery, the unit dose, as well as the active ingredients comprising the composition.

In some embodiments, a pharmaceutically acceptable excipient can comprise anhydrous calcium phosphate, dihydrate calcium phosphate, hydroxypropyl methylcellulose, croscarmellose sodium, GMO-free croscarmellose sodium, carbomers, magnesium aluminometasilicate, mannitol, povidone (PVP), crospovidone, sorbitol, dimethicone, sodium stearyl fumarate, sodium starch glycollate, hydroxypropylcellulose, native corn starch, modified corn starch, carrageenan, alginates, silicon dioxide, microcrystalline cellulose, carboxymethylcellulose sodium, alginates, carboxymethylcellulose (CMC), sodium carboxymethylcellulose (Na CMC), carbomers, natural gums, sorbitol, maltitol, glucose syrup, silicones, carbomers, fatty alcohols, alcohols, carbohydrates, petrolatum derivatives, butters, waxes, DMSO Procipient®, esters, fatty acids, oil-in-water (O/W) emulsifiers, water-in-oil (W/O) emulsifiers, silicas, fumed silicas, polysorbates, isopropyl myristate, cellulosic derivates, xanthan gum, propylenglycol, noveon AA-1 polycarbophyl, dimethyl isosorbate, polysilicone elastomer 1100, polysilicone elastomer 1148P, preservatives, flavors, colors, functional coatings, aesthetic coatings, a pharmaceutically acceptable salt of any of these, or any combination thereof.

In some embodiments, a pharmaceutically acceptable excipient can comprise acacia, acesulfame potassium, acetic acid, glacial, acetone, acetyl tributyl citrate, acetyl triethyl citrate, agar, albumin, alcohol, alginic acid, aliphatic polyesters, alitame, almond oil, alpha tocopherol, aluminum hydroxide adjuvant, aluminum oxide, aluminum phosphate adjuvant, aluminum stearate, ammonia solution, ammonium alginate, ascorbic acid, ascorbyl palmitate, aspartame, attapulgite, bentonite, benzalkonium chloride, benzethonium chloride, benzoic acid, benzyl alcohol, benzyl benzoate, boric acid, bronopol, butylated hydroxyanisole, butylated hydroxytoluene, butylparaben, calcium alginate, calcium carbonate, calcium phosphate, dibasic anhydrous, calcium phosphate, dibasic dihydrate, calcium phosphate, tribasic, calcium stearate, calcium sulfate, canola oil, carbomer, carbon dioxide, carboxymethylcellulose calcium, carboxymethylcellulose sodium, carrageenan, castor oil, castor oil, hydrogenated, cellulose (e.g. microcrystalline, powdered, silicified microcrystalline, acetate, acetate phthalate) ceratonia, cetostearyl alcohol, cetrimide, cetyl alcohol, cetylpyridinium chloride, chitosan, chlorhexidine, chlorobutanol, chlorocresol, chlorodifluoroethane, chlorofluorocarbons, chloroxylenol, cholesterol, citric acid monohydrate, colloidal silicon dioxide, coloring agents, copovidone, corn oil, cottonseed oil, cresol, croscarmellose sodium, crospovidone, cyclodextrins, cyclomethicone, denatonium benzoate, dextrates, dextrin, dextrose, dibutyl phthalate, dibutyl sebacate, diethanolamine, diethyl phthalate, difluoroethane, dimethicone, dimethyl ether, dimethyl phthalate, dimethyl sulfoxide, dimethylacetamide, disodium edetate, docusate sodium, edetic acid, erythorbic acid, erythritol, ethyl acetate, ethyl lactate, ethyl maltol, ethyl oleate, ethyl vanillin, ethylcellulose, ethylene glycol palmitostearate, ethylene vinyl acetate, ethylparaben, fructose, fumaric acid, gelatin, glucose, glycerin, glyceryl behenate, glyceryl monooleate, glyceryl monostearate, glyceryl palmitostearate, glycofurol, guar gum, hectorite, heptafluoropropane, hexetidine, hydrocarbons, hydrochloric acid, hydroxyethyl cellulose, hydroxyethylmethyl cellulose, hydroxypropyl cellulose, hydroxypropyl cellulose, low-substituted, hydroxypropyl starch, hypromellose, hypromellose acetate succinate, hypromellose phthalate, honey, imidurea, inulin, iron oxides, isomalt, isopropyl alcohol, isopropyl myristate, isopropyl palmitate, kaolin, lactic acid, lactitol, lactose, anhydrous, lactose, monohydrate, lactose, spray-dried, lanolin, lanolin alcohols, lanolin, hydrous, lauric acid, lecithin, leucine, linoleic acid, macrogol hydroxystearate, magnesium aluminum silicate, magnesium carbonate, magnesium oxide, magnesium silicate, magnesium stearate, magnesium trisilicate, malic acid, maltitol, maltitol solution, maltodextrin, maltol, maltose, mannitol, medium-chain triglycerides, meglumine, menthol, methylcellulose, methylparaben, mineral oil, mineral oil, light, mineral oil and lanolin alcohols, monoethanolamine, monosodium glutamate, monothioglycerol, myristic acid, neohesperidin dihydrochalcone, nitrogen, nitrous oxide, octyldodecanol, oleic acid, oleyl alcohol, olive oil, palmitic acid, paraffin, peanut oil, pectin, petrolatum, petrolatum and lanolin alcohols, phenol, phenoxyethanol, phenylethyl alcohol, phenylmercuric acetate, phenylmercuric borate, phenylmercuric nitrate, phosphoric acid, polacrilin potassium, poloxamer, polycarbophil, polydextrose, polyethylene glycol, polyethylene oxide, polymethacrylates, poly(methyl vinyl ether/maleic anhydride), polyoxyethylene alkyl ethers, polyoxyethylene castor oil derivatives, polyoxyethylene sorbitan fatty acid esters, polyoxyethylene stearates, polyvinyl acetate phthalate, polyvinyl alcohol, potassium alginate, potassium benzoate, potassium bicarbonate, potassium chloride, potassium citrate, potassium hydroxide, potassium metabisulfite, potassium sorbate, povidone, propionic acid, propyl gallate, propylene carbonate, propylene glycol, propylene glycol alginate, propylparaben, 2-pyrrolidone, raffinose, saccharin, saccharin sodium, saponite, sesame oil, shellac, simethicone, sodium acetate, sodium alginate, sodium ascorbate, sodium benzoate, sodium bicarbonate, sodium borate, sodium chloride, sodium citrate dihydrate, sodium cyclamate, sodium hyaluronate, sodium hydroxide, sodium lactate, sodium lauryl sulfate, sodium metabisulfite, sodium phosphate, dibasic, sodium phosphate, monobasic, sodium propionate, sodium starch glycolate, sodium stearyl fumarate, sodium sulfite, sorbic acid, sorbitan esters (sorbitan fatty acid esters), sorbitol, soybean oil, starch, starch (e.g. pregelatinized, sterilizable maize), stearic acid, stearyl alcohol, sucralose, sucrose, sugar, compressible, sugar, confectioner's, sugar spheres, sulfobutylether b-cyclodextrin, sulfuric acid, sunflower oil, suppository bases, hard fat, talc, tartaric acid, tetrafluoroethane, thaumatin, thimerosal, thymol, titanium dioxide, tragacanth, trehalose, triacetin, tributyl citrate, triethanolamine, triethyl citrate, vanillin, vegetable oil, hydrogenated, water, wax, anionic emulsifying, wax (e.g. carnauba, cetyl esters, microcrystalline, nonionic emulsifying, white, yellow), xanthan gum, xylitol, zein, zinc acetate, zinc stearate, or any combination thereof.

In some embodiments, a pharmaceutically acceptable excipient can comprise a carbohydrate, an alginate, povidone, a carbomer, a flavor, a natural gum, a silicone, an alcohol, a butter, a wax, a fatty acid, a preservative, a pharmaceutically acceptable salt of any of these, or any combination thereof. In some embodiments, a pharmaceutically acceptable excipient can comprise a carbohydrate. In some embodiments, the carbohydrate can comprise lactose, microcrystalline cellulose, cellulose, mannitol, sorbitol, starch, starch glycolate, hydroxypropyl methylcellulose, hydroxypropyl methylcellulose acetate succinate, a cyclodextrin, maltodextrin, croscarmellose sodium, corn starch, carrageenan, sorbitol, maltitol, glucose, a pharmaceutically acceptable salt of any of these, or any combination thereof.

In some aspects, the weigh to weight ratio of: a) the particles of the pharmaceutically acceptable excipient and b) the active ingredient particles ranges from about 1:1 to about 10000:1. In some aspects, the weight to weight ratio of: a) the particles of the pharmaceutically acceptable excipient and b) the active ingredient particles ranges from about 1:1 to about 20:1, about 1:1 to about 15:1, about 1:1 to about 10:1, about 1:1 to about 5:1, about 1:1 to about 2:1, about 2:1 to about 20:1, about 2:1 to about 15:1, about 2:1 to about 10:1, about 2:1 to about 5:1, about 5:1 to about 20:1, about 5:1 to about 15:1, about 5:1 to about 10:1, about 10:1 to about 15:1, about 10:1 to about 20:1, about 15:1 to about 20:1, about 18:1 to about 25:1, or about 25:1 to about 30:1. In some aspects, the weight to weight ratio of: a) the particles of the pharmaceutically acceptable excipient and b) the active ingredient particles may be about: 1:1, 2:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, or 30:1 In some aspects, the weight to weight ratio of: a) the particles of the pharmaceutically acceptable excipient and b) the active ingredient particles ranges from about 1:1 to about 1:10, about 1:1 to about 1:8, about 1:1 to about 1:5, about 1:1 to about 1:2, about 1:2 to about 1:10, about 1:2 to about 1:8, about 1:2 to about 1:5, about 1:5 to about 1:10, about 1:5 to about 1:8, about 1:8 to about 1:10.

### Carriers

In some instances, a pharmaceutically acceptable carrier or diluent can comprise water. In some embodiments, the water can be sterile. In some embodiments, the water can contain a buffer, a carbohydrate, a salt, a pH adjuster, or any combination of these. Simple sugars such as mannitol, sucrose, glucose, or trehalose may be added to inhibit peptide or polypeptide aggregation, in amounts from 1 to 50 mgs/ml. Citrate can be used as a buffer. In some instances, sodium chloride and phosphate salts may or may not be employed. Larger polysaccharides may also be used to enhance stability.

In certain instances, a carrier may refer to reagents, cells, compounds, materials, compositions, dosage forms, or any combination thereof that can be compatible with agents that can be administered therapeutically. In some cases, a carrier can be suitable for use in contact with a tissue of a subject. In some cases, a carrier may not have a toxicity, an irritation, an allergic response, or any combination thereof. A carrier that may be suitable for use can include a liquid, a solid material (e.g., a pill, or a suppository) or any combination thereof. In some cases, a carrier can be designed to resist degradation within the body (non-biodegradable) or they may be designed to degrade within the body (biodegradable). A biodegradable material can further be bioresorbable or bioabsorbable. In some cases, a biodegradable material can be degraded and eliminated from the body by conversion into other materials or breakdown and elimination through natural pathways.

### Oral Bioavailability

In some instances, the polypeptides, derivatives thereof, or salts of any of these can be orally bioavailable. The percent oral bioavailability can be at least, at least about, or about: 5%, 10% 20%, 30%, 40%, 50%, 60% 70%, 80%, 90%, or 95%, or 100%.

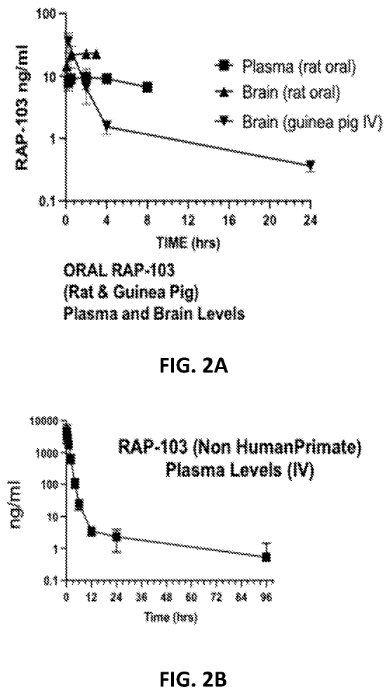

For example, in some studies, RAP-103 quickly entered the brain by oral, and IV dosing in rodents and non-human primates (Rhesus macaque). The non-human primates showed oral bioavailability of 88%. In some studies, RAP-103 preferentially entered the brain by oral compared to IV dosing, a feature that supports its use in the treatment of pain. (FIG. 2A).

In some instances, the polypeptides, derivatives, salts of any of these, can be administered to a subject, who can be a subject in need thereof. In some embodiments, the subject has peripheral neuropathy. In some embodiments, the subject can be a human, can be a male, or can be a female. In some instances, the subject can be under 18 years of age. In some instances, the subject can be over 18 years of age. In some instances, the subject can range from about 1 year of age to about 120 years of age.

### Administration

In some embodiments, the terms “administer,” “administering”, “administration,” and the like, as used herein, can refer to methods that can be used to enable delivery of compounds, polypeptides, derivatives thereof, or salts of any of these, or compositions described herein, to the desired site of biological action. In some cases, delivery can include injection, inhalation, catheterization, gastrostomy tube administration, intravenous administration, intraosseous administration, ocular administration, otic administration, topical administration, transdermal administration, local administration, oral administration, rectal administration, nasal administration, intravaginal administration, intracavernous administration, transurethral administration, buccal administration, sublingual administration, or a combination thereof. Delivery can include direct application to the affect tissue or region of the body. Delivery can include a parenchymal injection, an intra-thecal injection, an intra-ventricular injection, or an intra-cisternal injection. A composition provided herein can be administered by any method. A method of administration can be by intraarterial injection, intracerebroventricular injection, intracisternal injection, intramuscular injection, intraorbital injection, intraparenchymal injection, intraperitoneal injection, intraspinal injection, intrathecal injection, intravenous injection, intraventricular injection, stereotactic injection, subcutaneous injection, epidural, or any combination thereof. Delivery can include parenteral administration (including intravenous, subcutaneous, intrathecal, intraperitoneal, intramuscular, intravascular or infusion administration). In some embodiments, delivery can comprise a nanoparticle, a viral vector, a viral-like particle, a liposome, an exosome, an extracellular vesicle, a microrobot, a microneedle, an implant, or a combination thereof. In some cases, delivery can be from a device. In some instances, delivery can be administered by a pump, an infusion pump or a combination thereof. In some cases, delivery can be by an enema, an eye drop, a nasal spray, an ear drop, or any combination thereof. In some cases, delivery can comprise an inhaler, a diffuser, a nebulizer, or a combination thereof. Delivery can include topical administration (such as a lotion, a cream, a patch, a gel, a spray, a drip, a liquid formulation, an ointment) to an external surface of a surface, such as a skin. In some instances, a subject can administer the composition in the absence of supervision. In some instances, a subject can administer the composition under the supervision of a medical professional (e.g., a physician, nurse, physician's assistant, orderly, hospice worker, etc.). In some cases, a medical professional can administer the composition. In some cases, the subject can administer the composition.

In some embodiments, administering can be performed at least about: 1 time per day, 2 times per day, 3 times per day, 4 times per day, 5 times per day, 6 times per day or more than 6 times per day. In some cases, administering can be performed daily, weekly, monthly, or as needed. In some embodiments, administering can be conducted one, twice, three, or four times per day. In some cases, administration can be provided by a subject (e.g. the patient), a health care provider, or both.

Administration or application of a composition disclosed herein can be performed for a treatment duration of at least about at least about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 150, 200, 300, 400, 500, 600, 700, 800, 900, or 1000 days consecutive or nonconsecutive days. In some cases, a treatment duration can be from about 1 to about 30 days, from about 2 to about 30 days, from about 3 to about 30 days, from about 4 to about 30 days, from about 5 to about 30 days, from about 6 to about 30 days, from about 7 to about 30 days, from about 8 to about 30 days, from about 9 to about 30 days, from about 10 to about 30 days, from about 11 to about 30 days, from about 12 to about 30 days, from about 13 to about 30 days, from about 14 to about 30 days, from about 15 to about 30 days, from about 16 to about 30 days, from about 17 to about 30 days, from about 18 to about 30 days, from about 19 to about 30 days, from about 20 to about 30 days, from about 21 to about 30 days, from about 22 to about 30 days, from about 23 to about 30 days, from about 24 to about 30 days, from about 25 to about 30 days, from about 26 to about 30 days, from about 27 to about 30 days, from about 28 to about 30 days, from about 29 to about 30 days, from about 1 to about 90 days, from about 30 day to about 90 days, from about 60 days to about 90 days, from about 30 days to about 180 days, or from about 90 days to about 190 days.

Administration or application of a composition disclosed herein can be performed for a treatment duration of at least about 1 week, at least about 1 month, at least about 1 year, at least about 2 years, at least about 3 years, at least about 4 years, at least about 5 years, at least about 6 years, at least about 7 years, at least about 8 years, at least about 9 years, at least about 10 years, at least about 15 years, at least about 20 years, or for life. Administration can be performed repeatedly over a lifetime of a subject, such as once a month or once a year for the lifetime of a subject. Administration can be performed repeatedly over a substantial portion of a subject's life, such as once a month or once a year for at least about 1 year, 5 years, 10 years, 15 years, 20 years, 25 years, 30 years, or more.

Administration or application of composition disclosed herein can be performed at least 1, 2, 3, 4, 5, 6, 7, 8, 9, 10, 11, 12, 13, 14, 15, 16, 17, 18, 19, 20, 21-, 22-, 23-, or 24-times a in a 24-hour period. In some cases, administration or application of a composition disclosed herein can be performed continuously throughout a 24-hour period, for example, when an implant can be used for administration. In some cases, administration or application of composition disclosed herein can be performed at least 1, 2, 3, 4, 5, 6, 7, 8, 9, 10, 11, 12, 13, 14, 15, 16, 17, 18, 19, 20, or 21 times a week. In some cases, administration or application of composition disclosed herein can be performed at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, or 90 times a month. In some cases, a composition can be administered as a single dose or as divided doses. In some cases, the compositions described herein can be administered at a first time point and a second time point. In some cases, a composition can be administered such that a first administration can be administered before the other with a difference in administration time of 1 hour, 2 hours, 4 hours, 8 hours, 12 hours, 16 hours, 20 hours, 1 day, 2 days, 4 days, 7 days, 2 weeks, 4 weeks, 2 months, 3 months, 4 months, 5 months, 6 months, 7 months, 8 months, 9 months, 10 months, 11 months, 1 year or more.

In some embodiments, administering can be performed for about: 1 day to about 8 days, 1 week to about 5 weeks, 1 month to about 12 months, 1 year to about 3 years, 3 years to about 10 years, 10 years to about 50 years, 25 years to about 100 years, or 50 years to about 130 years.

In some embodiments, a subject can be from about 1 day to about 10 months old, from about 9 months to about 24 months old, from about 1 year to about 8 years old, from about 5 years to about 25 years old, from about 20 years to about 50 years old, from about 40 years to about 80 years old, or from about 50 years to about 130 years old.

In some embodiments, the composition can be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some cases, the polypeptide or the derivative thereof or the salt of any of these can be administered in a pharmaceutical composition, which can be in unit dose form. In some instances, the amount of the polypeptide, or the derivative thereof, or the salt of any of these can be dosed in an amount ranging from about 0.0001 mg/kg of body weight of the subject to about 1000 g/kg of body weight of the subject; the dosage can be, for example, based on mg of polypeptide, derivative thereof, or salt thereof, per kg of subject body weight, can be about: 0.0001, 0.001, 0.01. 0.1, 0.2, 0.3, 0.4, 0.5, 0.6, 0.7, 0.8, 0.9 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, or 1000 g/kg of subject body weight.

In some instances, the amount of polypeptide, derivative thereof, or salt of any of these, which can be a pharmaceutically acceptable salt, that is dosed to the patient can range from 0.00001 mg to 1000 g; the dosage can be for example, about: 0.0001, 0.001, 0.01, 0.1, 1, 2, 3, 4, 5, 6, 7, 8, 9, 10, 15, 20, 25, 30, 35, 40, 45, 50, 55, 60, 65, 70, 75, 80, 85, 90, 95, 100, 200, 300, 400, 500, 600, 700, 800, 900, or 1000 g.

In some instances, a composition or pharmaceutical composition may be in the form of a capsule, a tablet, a gummy, an oil, a liquid, a tincture, a lotion, a cream, a balm, a candy, a chocolate, a food, a drink, an oil, a suppository, a liquid for injection, which can be, for example, an intra venous liquid, an intra muscular liquid, or subcutaneous liquid; a syrup or any combination thereof.

### Diagnoses

In some embodiments, a method can further comprise diagnosing a subject as having the disease. In some embodiments, a diagnosing can comprise employing an in vitro diagnostic. In some embodiments, the in vitro diagnostic can be a companion diagnostic.

In some embodiments, a diagnosis can comprise a physical examination, a radiological image, a blood, body fluid or tissue test, an antibody test, or any combination thereof. The diagnostic analyte can be a cytokine, such as a proinflammatory cytokine or a chemokine, or their receptors.

In some embodiments, a diagnosis can comprise a radiological image and the radiological image can comprise: a computed tomography (CT) image, an X-Ray image, a magnetic resonance image (MRI), an ultrasound image, or any combination thereof. Imaging markers of brain inflammation such as 18F-FEPPA, a TSPO ligand, may be used to support diagnoses or response to treatment. TSPO in some instances can mean translocator protein.

In some aspects, a method may further comprise diagnosing a subject as having the disease. In some aspects, a diagnosing may comprise employing an in vitro diagnostic. In some aspects, the in vitro diagnostic may be a companion diagnostic.

In some aspects, a diagnosis may comprise a physical examination, a radiological image, a blood test, an antibody test, or any combination thereof. In some aspects, a diagnosis may comprise a radiological image and the radiological image may comprise: a computed tomography (CT) image, an X-Ray image, a magnetic resonance image (MRI), an ultrasound image, or any combination thereof.

### Kits

Also disclosed herein are kits comprising the pharmaceutical composition contained at least in part in packaging. Also disclosed herein are methods of making kits comprising a pharmaceutical composition contained at least in part in packaging.

### Methods of Treatment

Also disclosed herein are methods of treating a disease comprising treating the disease or condition by administering a therapeutically effective amount of the pharmaceutical composition.

In certain aspects, the condition is a peripheral neuropathy. In some instances, the peripheral neuropathy is a chemotherapy-induced peripheral neuropathy (CIPN). In some instances, the peripheral neuropathy is an amyloid peripheral neuropathy. In some instances, the peripheral neuropathy is an inflammatory peripheral neuropathy. In some instances, the peripheral neuropathy is an idiopathic peripheral neuropathy. In some instances, the peripheral neuropathy is a hereditary peripheral neuropathy such as Charcot-Marie-Tooth disease or Hereditary neuropathy with liability to pressure palsies (HNPP). In some instances, the peripheral neuropathy is a peripheral neuropathy due to exposure to environmental toxins such as mercury, arsenic, and thallium. In some instances, the peripheral neuropathy is alcoholism related peripheral neuropathy. In some instances, the peripheral neuropathy is an infection related peripheral neuropathy such as a Lyme disease infection, HIV infection, leprosy, herpes zoster infection, hepatitis B infection, or Hepatitis C infection. In some instances, the peripheral neuropathy is associated with an autoimmune disorder such as sarcoidosis, Guillain-Barré Syndrome/Acute Inflammatory Demyelinating Polyneuropathy (AIDP), Chronic Inflammatory Demyelinating Polyneuropathy (CIDP), Polyarteritis Nodosa (PAN), Rheumatoid Arthritis, Systemic Lupus Erythematosus (Lupus), Sjögren's Syndrome, Celiac Disease, or Multifocal Motor Neuropathy (MNN). In some instances, the peripheral neuropathy is associated with a vitamin deficiency. In some instances, the peripheral neuropathy is associated with kidney failure. In some instances, the peripheral neuropathy is a Bell's palsy related peripheral neuropathy.

In certain instances, the peripheral neuropathy is due to administration of a pharmaceutical drug such as a drug used for heart treatment and/or high blood pressure such as amiodarone, hydralazine, perhexiline, combinations thereof or derivatives thereof.

In certain instances, the peripheral neuropathy is due to administration of a pharmaceutical drug such as a drug used as a chemotherapy drug against a hyperproliferative disease such as cancer. These drugs may have been administered in various combinations or derivative forms. In such instances, the chemotherapy drug may be a platinum drug, such as cisplatin, oxaliplatin, combinations thereof or derivatives thereof. In some instances, the chemotherapy drug may be a taxane such as docetaxel, paclitaxel, combinations thereof or derivatives thereof. In some instances, the chemotherapy drug may be a plant alkaloid such as vincristine, vinblastine, combinations thereof or derivatives thereof. In some instances, the chemotherapy drug may be an epothilone such as ixabepilone or a derivative thereof. In some instances, the chemotherapy drug may be a proteasome inhibitor such as bortezomib or a derivative thereof. In some instances, the chemotherapy drug is an immunomodulatory drug such as thalidomide or a derivative thereof. In some instances, the chemotherapy drug is a pyrimidine analog such as 5-fluorouracil or a derivative thereof.

In certain instances, the peripheral neuropathy is due to administration of a pharmaceutical drug such as an anti-infective. In certain instances, the drug is suramin or a derivative thereof. In certain instances, the drug is dapsone or a derivative thereof. In certain instances, the drug is nitrofurantoin or a derivative thereof. In certain instances, the drug is metronidazole or a derivative thereof.

### Co-Therapies

In some aspects, a method may further comprise administering a second therapy to the subject. In some aspects, a second therapy may comprise acetaminophen, an opioid, prednisone, cortisone, a gabapentinoid, a voltage gated sodium channel inhibitor, an anti-nerve growth factor, a salt of any of these, or any combination thereof. In some instances, the second therapy may comprise a nonsteroidal anti-inflammatory drug and the nonsteroidal anti-inflammatory drug may comprise naproxen, ibuprofen, acetaminophen, aspirin a salt of any of these, or any combination thereof. In some cases, a second therapy can be administered concurrently or consecutively with a peptide disclosed herein.

In some aspects, the composition may be administered as needed, or for: one day, two days, three days, four days, five days, six days, a week, two weeks, three weeks, a month, two months, three months, four months, five months, six months, seven months, eight months, nine months, ten months, eleven months, a year, or chronically.

In some aspects, the composition may be administered so that the active ingredient or the pharmaceutically acceptable salt thereof in the unit dose ranges from about: 500 μg (micrograms) to about 1000 mg, 10 μg to about 50 μg, 40 μg to about 90 μg, 80 μg to about 120 μg, 100 μg to about 150 μg, 140 μg to about 190 μg, 150 μg to about 220 μg, 200 μg to about 250 μg, 240 μg to about 300 μg, 290 μg to about 350 μg, 340 μg to about 410 μg, 400 μg to about 450 μg, 440 μg to about 500 μg, 500 μg to about 700 μg, 600 μg to about 900 μg, 800 μg to about 1 mg, 1 mg to about 5 mg, 1 mg to about 10 mg, 5 mg to about 15 mg, 12 mg to about 25 mg, 20 mg to about 50 mg, 40 mg to about 80 mg, 70 mg to about 100 mg, 90 mg to about 150 mg, 125 mg to about 250 mg, 200 mg to about 500 mg, 400 mg to about 750 mg, 700 mg to about 900 mg, or from about 850 mg to about 1000 mg. In some cases, the unit dose range may be more than about: 10 μg, 25 μg, 50 μg, 75 μg, 100 μg, 150 μg, 200 μg, 220μ g, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, the unit dose range may be less than about: 10 μg, 25 μg, 50 μg, 75 μg, 100 μg, 150 μg, 200 μg, 220 μg, 250 μg, 300 μg, 350 μg, 400 μg, 450 μg, 500 μg, 550 μg, 600 μg, 650 μg, 700 μg, 750 μg, 800 μg, 850 μg, 900 μg, 950 μg, 1000 μg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, or 25 mg. In some cases, epinephrine or a salt thereof may be administered in a unit dose form of about 0.22 mg. In some cases, epinephrine or a salt thereof may be administered in a unit dose form of about 0.10, 0.20, 0.30, 0.40 or 0.50 mg.

### EXAMPLES

The following examples are included for illustrative purposes only and are not intended to limit the scope of the disclosure.

### Example 1

The synthetic process used for manufacture of RAP-103 Acetate Salt involves the following steps which follow the Merrifield FMOC synthesis method SPPS (Solid phase Peptide Synthesis), using the common commercially available coupling reagents, resins, and deblocking reagents:
Step 1: peptide synthesis: solid phase peptide synthesis (SPPS) of the protected peptide
Step 2: cleavage and deprotection: trifluoroacetic acid (TFA) cleavage of the protecting groups from the peptide and cleavage of the peptide from the resin
Step 3: purification and in-process lyophilization: peptide purification and in-process lyophilization of the peptide
Step 4: ion exchange (salt exchange) and final lyophilization: ion exchange (salt exchange) from TFA to acetate salt and Final lyophilization.

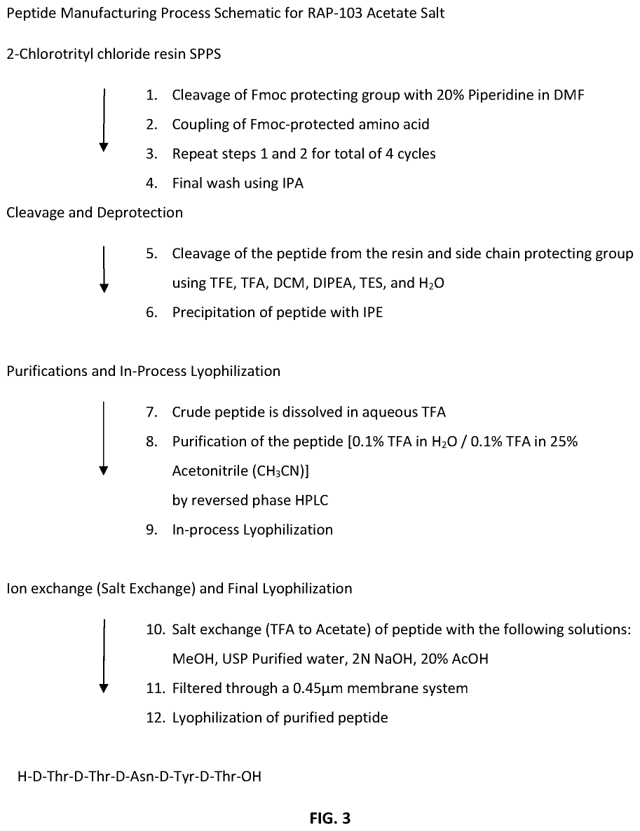

A schematic of a synthesis method for RAP-103 is shown in FIG. 3 and comprises the following steps: 2-chlorotrityl chloride resin SPPS, cleavage and deprotection, urifications and in-process lyophilization, ion exchange (salt exchange) and final lyophilization. The following components are equipment and components were used for synthesis.

### Reaction Vessel

The synthesis was carried out at room temperature in a custom-designed glass vessel, with the bottom part comprising a fritted disk of coarse porosity. The size of the reactor is dependent on the amount of polymer to be used for the synthesis. The reactor was designed to assist in the addition of amino acid derivatives, solvents and reagents, as required. The reaction vessel was equipped with a mechanical stirrer to allow for efficient mixing of the peptide-resin. No components of the equipment or utensils utilized for the synthesis process were composed of materials that can cause adulteration of the product. Solid phase support: 2-Chlorotrityl chloride resin was used for the synthesis.

### Protected Amino Acids

In solid phase peptide synthesis, the reactive functional groups of the amino acids were protected to avoid undesirable side reactions. The protecting groups were of two natures: acid labile and base labile. The base labile protecting group was used to block the α-amino group during the coupling reaction and was removed in the deblocking step, to allow the introduction of the next amino acid in the sequence. Fmoc (9-Fluorenylmethyloxycarbonyl) was used as the base labile a-amino protecting group. The acid labile protecting group was used to protect the side-chain reactive functional groups of the amino acids during synthesis and must be resistant to the deblocking mixture (20% piperidine in DMF). Following the peptide synthesis, these protecting groups were removed by strong acid (aqueous trifluoroacetic acid with scavengers). The acid-labile protecting groups for this process are t-butyl (tBu), trityl (Trt).

The following amino acids were used in the synthesis of RAP-103 Acetate Salt: Fmoc-D-Tyr (tBu)-OH, Fmoc-D-Asn (Trt)-OH, and Fmoc-D-Thr (tBu)-OH.

### Step 1: Peptide Synthesis:

Resin Loading-2-Chlorotrityl chloride (CTC) resin was activated with Acetyl chloride (AcCl) and then treated with Fmoc-D-Thr (tBu)-OH and Diisopropylethylamine (DIPEA) followed by a solution mixture of Dichloromethane (DCM), Methanol (MeOH) and Diisopropylethylamine (DIPEA).

The solid phase peptide synthesis by Fmoc strategy can be divided into the following steps:

### Fmoc Deprotection:

During the deprotection step, the base-labile temporary protecting group (Fmoc) was cleaved from the α amino function of the N-terminal amino acid on the growing peptide chain by treating the resin twice with a solution of 20% piperidine in dimethylformamide (DMF). Two deprotection treatments were performed, the 1st deprotection stir time was approximately 10 minutes, and the 2nd deprotection stir time was approximately 30 minutes.

### Wash Cycle:

The wash steps were performed to eliminate excess reagents used in the preceding step. The solvents selected for each step were carefully chosen to ensure that there is no risk of introducing an undesirable side reaction while eliminating the excess of reagents as efficiently as possible. The duration of each wash step was timed to allow for thorough contact of the peptide-resin with the solvent and to provide ample time for extraction of the reagents. DMF was used after deblocking as well as after coupling because it has excellent solubilizing and swelling properties for all reagents used in the coupling step. Conversely, isopropanol (IPA) was utilized after coupling reaction because it shrinks the resin, which also aids in removal of excess solvents and reagents.

### Activation and Coupling:

During the activation and coupling steps, the deprotected a-amino group is acylated by the next activated amino acid in the sequence. The reagents used to accomplish acylation were carefully selected to create optimal reaction conditions and easy elimination of the excess reagents at the end of the coupling reaction.

Activation of Fmoc-Tyr (tBu)-OH was performed by dissolving the protected amino acid with coupling reagents 1-H-Benzotriazolium, 1-[bis(dimethylamino)methylene]-5-chloro-tetrafluoroborate (1-),3-oxide (TCTU) and diisopropylethylamine (DIPEA) in DMF. The solution of activated amino acid was then added to the peptide-resin. The mixture was stirred at room temperature for 20 minutes and then DIPEA in DMF was added in it. The mixture was allowed to react for approximately 160 minutes.

Activation of the remaining amino acid derivatives was performed by dissolving the protected amino acid with coupling reagents Oxima (Oxymapure) in DMF and 1,3-Diisopropylcarbodiimide (DIC). The solution of activated amino acid was then added to the peptide-resin. The suspension was stirred at room temperature for 20 minutes, after which a second aliquot of DIC was added to the reaction mixture. The mixture was stirred and allowed to react for approximately 160 minutes.

### Recoupling and Acetylation:

After a minimal reaction time of one hour, the presence of remaining unreacted amino groups was monitored using the qualitative TNBS (trinitrobenzenesulfonic acid) test or the Ninhydrin test. The TNBS test is performed adding a few drops of trinitrobenzenesulfonic acid to the peptide-resin in a test tube sample and allowing the two to react for three minutes. The presence of free amino groups causes a colored reaction; orange-colored beads indicate incomplete coupling and the presence of unreacted amine. Similarly, in the Ninhydrin test, a few drops of the Ninhydrin reagents were added to a sample of the peptide-resin in a small test tube. Blue-stained resin beads indicate the presence of unreacted amine.

If some residual amino groups were detected by either of the above-mentioned tests, the coupling reaction was repeated using half the amount of amino acid derivative required for the first coupling reaction. The Ninhydrin test was performed each time coupling takes place to visualize the presence of unreacted a-amino functions. No recoupling reactions were required during the manufacture of RAP-103 Acetate Salt lot 1000008388.

If unreacted a-amino functions are still present after recoupling, they are acetylated using acetic anhydride to avoid undesirable deletion sequences in the next cycle. No acetylation reactions were required during the manufacture of RAP-103 Acetate Salt lot 1000008388.

After coupling of the last amino acid in the sequence was completed, the peptide-resin was thoroughly washed using Isopropyl Alcohol (IPA) and weighed.

### Step 2: Cleavage and Deprotection:

During the cleavage operation, the peptide was detached from the resin with concomitant cleavage of the side chain protecting groups. This was accomplished by the treatment of the peptide-resin with trifluoroacetic acid (TFA) in the presence of scavengers and Trifluoroethanol (TFE) in TFA and Dichloromethane (DCM). Triethylsilane (TES) and water acted as scavengers and were used to provide a protonated cleavage environment which in turn gives higher quality crude. Following the cleavage operation, the peptide was precipitated using cooled isopropyl ether (IPE), and filtered using a Buchner funnel with filter paper. The precipitated peptide was washed with IPE and dried in a vacuum oven at room temperature. After drying was completed, the crude peptide was weighed and recorded.

### Step 3: Purification and In-process Lyophilization:

The purification is performed by preparative Reversed Phase High Performance Chromatography (RP-HPLC).

### Equipment:

The purification equipment was based on the principle of compression in which the chromatographic support is packed in a compression module. A constant pressure was applied to the column. Different column sizes are available and the choice of which to use is based on the amount of material to be processed. The solvents were delivered through pumps and the necessary gradients are created manually or with an automatic gradient maker.

### Nature of Support:

The purification of crude peptide was accomplished by preparative HPLC using reversed phase material as the support. The reversed phase material consists of a silica gel coated with aliphatic chains; the free remaining silanol groups have been end-capped to avoid undesirable ionic interaction/binding between the mixture to be purified and the support. The separation was based on the hydrophobic interaction between the peptide and the resin support. The use of different buffer systems in subsequent purification steps also improves the separation efficiency.

### Purification Using an Aqueous TFA/Acetonitrile Buffer Gradient:

A typical purification run consists of three steps: equilibration Luna C18 column, loading and elution of the product, and washing of the column to prepare it for the next run. Equilibration of the column was accomplished by washing it with aqueous TFA solution. The crude peptide was dissolved in an aqueous TFA solution, filtered and then was loaded onto the column. Product elution was achieved using a gradient of aqueous TFA and acetonitrile (CH3CN) buffer solutions. After product elution, the column was washed with aqueous acetonitrile to check the absence of product. The quality of each different fraction that were collected as the peptide elutes from the column was monitored by analytical HPLC. The fractions, which met the acceptance criteria for purity, were pooled as the main pool and proceed to the next step.

### In-Process Lyophilization:

The main pool of the product from the TFA purification step was filtered through a 0.45 μm membrane filter and lyophilized.

### Step 4: Ion-Exchange (Salt Exchange) and Final Lyophilization:

### Ion-Exchange (Salt Exchange):

The Ion-exchange, also referred to as the salt exchange stage, converts the peptide into the required salt form (acetate salt). The preparation of ion-exchange resin (AMBERLITE IRN 78) was accomplished by washing the resin sequentially with methanol (MeOH), USP Water, 2N sodium hydroxide (NaOH), USP water, acetic acid (AcOH-20% in USP water) and USP water until neutrality. The peptide from in-process lyophilization step was dissolved in USP water and loaded onto already prepared Ion-exchange resin. After circulating for two hours, the peptide solution was eluted and collected in fraction collecting bottles. All fractions that met the establish criteria after analyzing with HPLC were collected and pooled together.

### Final Lyophilization

The main pool of the peptide solution from ion-exchange (salt exchange) step was filtered through 0.45 μm membrane filtration cap. The resulting filtrate (peptide solution) was lyophilized to obtain a bulk RAP-103 acetate salt peptide.

### Example 2

A peptide described herein can be administered by a pill or a capsule to a subject in need thereof. The pills or capsules contain excipients to enhance stability, dissolution, and absorption. Enteric coatings are applied to control delivery and maintain therapeutic levels. In another other example, liquid solutions in water or saline are prepared for IV, sub-cutaneous, or intra-muscular delivery. Reconstitution at the time of use extends the shelf-life. The weight/weight ratio of drug (active pharmaceutical peptide) to excipient can be 0.01 to 0.25. In some cases, the weight/weight ratio of drug (active pharmaceutical peptide) to excipient can be 0.005 to 0.5.

### Example 3

Animals (rats, guinea pigs, and non-human primates (Rhesus macaques; Macaca mulatta) were administered RAP-103 by oral gavage and IV routes. Blood samples were collected into K2EDTA MAP tubes, placed on wet ice, and processed to plasma (in a centrifuge set to maintain 2000 g, at 4° C. for 15 minutes) within 60 minutes of collection and were stored in a freezer set to maintain−80° C. until analysis. Samples were analyzed by using a qualified high performance liquid chromatography (HPLC) with mass spectrometric (MS/MS) detection to determine the concentrations of RAP-103. Giving dosage forms to animals shows that RAP-103 quickly entered the brain (rats and guinea pigs) by IV or oral gavage dosing and persisted at therapeutic levels for at least 24 hrs as shown in FIG. 2A. RAP-103 preferentially enters the brain compared to plasma levels. In non-human primates (rhesus monkeys) RAP-103 was dosed once on Day 1 by intravenous bolus injection at 1 mg/kg and plasma levels determined. The Cmax and AUC 0-T were comparable in male and female monkeys. RAP-103 was highly bioavailable at the 1 mg/kg dose level with absolute bioavailability value of 88% and 89% in females and males. Drug in plasma after a single IV dose (1 mg/Kg) was still measurable at anticipated therapeutic levels 96 hrs. post-dose in non-human primates as shown in FIG. 2B. There were no RAP-103-related changes noted in clinical observations or body weights over seven days indicating no acute toxicity.

### Example 4

The ability of RAP-103 was tested to treat chemotherapy induced peripheral neuropathy (CIPN) caused by paclitaxel in rodents. This assay uses the chemotherapeutic agent paclitaxel to elicit a chronic pain state. Once established, pain in these animals can persist for weeks.

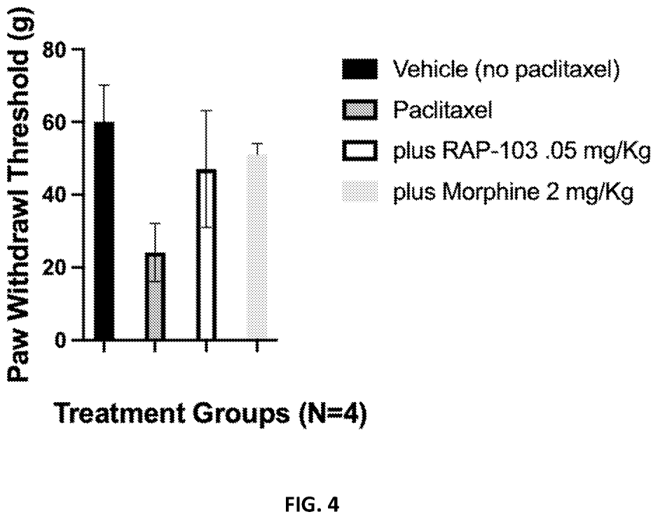

Rats were given i.p. injections of paclitaxel (2 mg/kg) on days 0, 2, 4, and 6 to induce a chronic pain state. Rats who exhibited stable mechanical allodynia were randomized to experimental groups (N=4 each) who received water (vehicle) or RAP-103 (0.05 mg/kg) by daily i.p. (intraperitoneal) injections for five days of exposure to RAP-103 on days 17 through 21 post paclitaxel treatment. On day 21 morphine (2 mg/Kg) was also administered to animals as a positive control. Pain was assessed 60 minutes after morphine or RAP-103 administration on the left rear paw on day 21 with a series of von Frey filaments with logarithmically incrementing stiffness applied perpendicular to the midplantar region of the hind paw. The paw withdrawal force (grams, g) threshold was determined. The 50% paw withdrawal threshold was determined using Dixon's up-down method and data are expressed as the Paw Withdrawal Threshold (g), Mean±S.E.M. As shown in FIG. 4, RAP-103 reduced chronic pain caused by repeated administration of paclitaxel to mimic the situation in human patients treated with chemotherapeutic agents.

While preferred aspects of the present disclosure have been shown and described herein, it will be obvious to those skilled in the art that such aspects are provided by way of example only. Numerous variations, changes, and substitutions will now occur to those skilled in the art without departing from the disclosure. It should be understood that various alternatives to the aspects of the disclosure described herein may be employed in practicing the methods presented in the disclosure. It is intended that the following claims define the scope of the disclosure and that methods and structures within the scope of these claims and their equivalents be covered thereby.

What is claimed is:

1. A method of treating a chemotherapy induced peripheral neuropathy (CIPN) in a subject in need thereof, the method comprising administering to the subject a therapeutically effective amount of a pharmaceutical composition to treat CIPN, wherein the pharmaceutical composition comprises:
(a) a polypeptide, a derivative thereof, or a salt thereof, wherein the polypeptide comprises at least five contiguous amino acids or derivatives thereof comprising the general formula: E-F-G-H-I, wherein:
E is D-Ser, D-Thr, D-Asn, D-Glu, D-Arg, D-Ile, or D-Leu, or a derivative of any of these;
F is D-Ser, D-Thr, D-Asp, or D-Asn, or a derivative of any of these;
G is D-Thr, D-Ser, D-Asn, D-Arg, D-Gln, D-Lys, or D-Trp, or a derivative of any of these;
H is D-Tyr, or a derivative thereof; and
I is D-Thr, D-Ser, D-Arg, or Gly, or a derivative of any of these.

2. The method of claim 1, wherein the polypeptide, the derivative thereof, or the salt thereof comprises at least eight contiguous amino acids or derivatives thereof, comprising the general formula A-B-C-E-F-G-H-I, and wherein:
A is D-Ala, or a derivative thereof;
B is D-Ser, or D-Thr, or a derivative of any of these;
C is D-Ser, or D-Thr, or a derivative of any of these;
E is D-Ser, D-Thr, D-Asn, D-Glu, D-Arg, D-Ile, or D-Leu, or a derivative of any of these;
F is D-Ser, D-Thr, D-Asp, or D-Asn, or a derivative of any of these;
G is D-Thr, D-Ser, D-Asn, D-Arg, D-Gln, D-Lys, or D-Trp, or a derivative of any of these;
H is D-Tyr, or a derivative thereof; and
I is D-Thr, D-Ser, D-Arg, or Gly or a derivative of any of these.

3. The method of claim 1 or claim 2, wherein the polypeptide or the salt thereof is D-Thr, D-Thr, D-Asn, D-Tyr, and D-Thr or a salt thereof.

4. The method of any one of claims 1-3, comprising the derivative of I, wherein I is esterified, glycosylated, or amidated at the C terminus.

5. The method of any one of claims 1-4, wherein the pharmaceutical composition is in unit dose form.

6. The method of any one of claims 1-5, wherein the pharmaceutical composition further comprises an excipient, a diluent, a carrier, or a combination thereof.

7. The method of any one of claims 1-6, wherein CIPN is accompanied or preceded by a nerve injury, an inflammation, a pain or a combination thereof.

8. The method of any one of claims 1-7, wherein the administering is daily, weekly, or monthly.

9. The method of any one of claims 1-8, wherein administering is once, twice, three, or four times per day.

10. The method of any one of claims 1-9, wherein the pharmaceutical composition is administered for about: one day, two days, three days, four days, five days, six days, one week, two weeks, three weeks, four weeks, five weeks, one month, two months, three months, four months, five months, six months, seven months, eight months, nine months, ten months, eleven months, one year, two years, or for life.

11. The method of any one of claims 1-10, wherein pharmaceutical composition comprises the polypeptide, the derivative thereof, or the salt thereof in an amount of from about 0.005 mg to about 1000 mg.

12. The method of any one of claims 1-11, wherein the pharmaceutical composition is administered by: an oral route, an injection route, a sublingual route, a buccal route, a rectal route, a vaginal route, an ocular route, an otic route, a nasal route, an internasal route, an inhalation route, a cutaneous route, a subcutaneous route, an intramuscular route, an intravenous route, a systemic route, a local route, a transdermal route, or any combination thereof.

13. The method of any one of claims 1-12, wherein the pharmaceutical composition is formulated for oral administration.

14. The method of claim 13, wherein the pharmaceutical composition is in a form of a pill or a liquid.

15. The method of any one of claims 1-14, wherein a second therapy is administered concurrently or consecutively.

16. The method of claim 15, wherein the second therapy comprises an gabapentinoid, an opioid, a voltage-gated sodium channel inhibitor, an anti-nerve growth factor, an nonsteroidal anti-inflammatory drug, aspirin, a corticosteroid, acetaminophen, a muscle relaxant, an anti-anxiety drug, an antidepressant, a cox-2 inhibitor, a local anesthetic, an anticonvulsant, a cannabinoid, an NMDA receptor antagonist, an α2-adrenergic receptor agonist or any combination thereof.

17. The method of any one of claims 15-16, wherein the pharmaceutical composition further comprises the second therapy.

18. The method of any one of claims 1-17, wherein the subject was diagnosed with the CIPN prior to the administration.

19. The method of any one of claims 1-18, wherein the subject was diagnosed with pain prior to the administration.

20. The method of any one of claims 18-19, wherein the diagnoses comprise an in vitro test, a physical exam, an imaging diagnostic or a combination thereof.

21. The method of any one of claims 1-20, wherein the CIPN is associated with administration of a platinum-based chemotherapy drug.

22. The method of claim 21, wherein the platinum-based chemotherapy drug is cisplatin, carboplatin, oxaliplatin, a salt of any of these, or a derivative of any of these.

23. The method of any one of claims 1-20, wherein the CIPN is associated with administration of a taxane.

24. The method of claim 23, wherein the taxane is docetaxel, paclitaxel, cabazitaxel, a salt of any of these, or a derivative of any of these.

25. The method of any one of claims 1-20, wherein the CIPN is associated with administration of an epothilone.

26. The method of claim 25, wherein the epothilone is ixabepilone or a salt thereof.

27. The method of any one of claims 1-20, wherein the CIPN is associated with administration of a plant alkaloid.

28. The method of claim 27, wherein the plant alkaloid is vinblastine, vincristine, vinorelbine, etoposide, a salt of any of these, or a derivative of any of these.

29. The method of any one of claims 1-20, wherein the CIPN is associated with administration of a thalidomide, a lenalidomide, a pomalidomide, a salt of any of these, or a derivative of any of these.

30. The method of any one of claims 1-20, wherein the CIPN is associated with administration of a proteasome inhibitor.

31. The method of claim 30, wherein the proteasome inhibitor is a bortezomib, a carfilzomib, a salt of any of these, or a derivative of any of these.

32. The method of any one of claims 1-31, wherein the subject is a mammal.

33. The method of claim 32, wherein the mammal is a human.

34. The method of claim 1, wherein the pharmaceutical composition treats CIPN at a dose 40-fold lower weight-to-weight than morphine in a rat animal model.


SEQUENCE LISTING (3 sequences)

SEQ ID NO: 1 - protein, 8 aa, Artificial Sequence
  Description of Artificial Sequence Synthetic peptide
ASTTTNYT

SEQ ID NO: 2 - protein, 5 aa, Artificial Sequence
  Description of Artificial Sequence Synthetic peptide
TTNYT

SEQ ID NO: 3 - protein, 8 aa, Artificial Sequence
  Description of Artificial Sequence Synthetic peptide
  C-term NH2
ASTTTNYT